(19) United States
(12) Patent Application Publication
(10) Pub. No.: US 2023/0390145 A1
(43) Pub. Date: Dec. 7, 2023

(54) COMPRESSION DEVICE

(71) Applicant: NATIONAL UNIVERSITY OF IRELAND, GALWAY, Galway (IE)

(72) Inventors: Aaron HANNON, Co. Mayo (IE); Bárbara OLIVEIRA, Co. Mayo (IE); Martin O'HALLORAN, Co. Mayo (IE)

(73) Assignee: NATIONAL UNIVERSITY OF IRELAND, GALWAY, Galway (IE)

(21) Appl. No.: 18/250,797

(22) PCT Filed: Oct. 27, 2021
(86) PCT No.: PCT/EP2021/079891

§ 371 Date: Apr. 27, 2023

(30) Foreign Application Priority Data
Oct. 27, 2020 (EP) 20204121.6

(51) Int. Cl. A61H 9/00; A61B 5/026; A61B 5/00

(52) U.S. Cl. CPC A61H 9/0078; A61B 5/0261; A61B 5/6803; A61B 5/4836; A61H 2201/165; A61H 2201/5071; A61H 2205/021

(57) ABSTRACT

A medical device comprising a bladder (or plurality of bladders), an attachment mechanism, a fluid inlet, a fluid outlet, a compressor, a pressure regulator system, and a perfusion sensor, which is compressed against intact tissue (unbroken skin or surface tissue in a cavity) for the purpose of minimising blood perfusion to prevent drug delivery to a non-target site.

### FIELD OF THE INVENTION

The invention relates to a pneumatic pressure device configured to reduce the likelihood of chemotherapy-induced side effects in a subject, including, but not limited to, chemotherapy-induced alopecia (CIA), infertility and peripheral neuropathy.

### BACKGROUND TO THE INVENTION

Chemotherapy is one of the most widely used treatments for cancer, with an estimated 9.8 million patients treated with chemotherapy regimens globally in 2018. Chemotherapy acts by targeting fast-growing, rapidly multiplying cells—a known characteristic of cancerous cells. This accounts for the effectiveness of chemotherapy in cancer therapy, but also for several side effects of chemotherapy treatment that result from systemic delivery of chemotherapy drugs.

Many non-cancerous cells ordinarily present in the body share the characteristic of being fast-dividing. This leads to several well recognised side effects of cancer treatment such as chemotherapy-induced alopecia (CIA), chemotherapy-induced peripheral neuropathy (CIPN), and chemotherapy-induced infertility (CII), among others. With usage of chemotherapy projected to grow by 53% by 2040, there is a pressing need to deal with the side effects of this core element of cancer treatment.

Chemotherapy-induced alopecia (CIA) affects at least 4.5 million people per year and is considered by patients as the most traumatic aspect of cancer treatment. CIA is an obvious physical symptom of cancer treatment, and it is a constant reminder to the patient of their disease, with negative psychological impacts on the patient. For example, 47% of female breast cancer patients consider CIA the most traumatic aspect of chemotherapy.

2.5 million women undergo treatments likely to cause CIA annually, with over 1 million of this group undergoing treatment for breast cancer, and a further 1.2 million for solid tumour treatments. A growing body of patients and clinicians have recognised the need for CIA treatment, leading to increased focus on developing preventative solutions.

Methods for preventing CIA have been on the market for over 20 years in the form of “Cold Cap” therapies, which use dry ice/gels and a head-covering cap to cause constriction of the scalp's blood vessels, preventing the destructive action of chemotherapy agents on the hair follicles. In the past number of years, “scalp cooling” technology has been developed, which is a more modern, FDA approved, machine-based cooling therapy. Using the same working principle, scalp cooling has become increasingly popular due to its reduced treatment times and relative simplicity of use compared to Cold Caps.

The cold caps and scalp cooling caps are typically a head-covering cap that is either packed with dry ice, in the case of a manual cap, or connected to a circulating coolant system in the case of a scalp cooler. The intended mechanism of action is that the reduced tissue temperature causes vasoconstriction in the local blood vessels, limiting blood flow to the hair follicle. Furthermore, the reduced temperature also causes a reduction in the metabolic rate of cells in the scalp region. This combined effect causes a reduced level of chemotherapy action on the hair follicle and thus reduced hair loss. Examples of the scalp cooling technologies can be found in published pamphlet WO 2019/222044 and U.S. Pat. No. 10,478,637 B2.

U.S. Pat. No. 7,744,640 describes a wound treatment device which is conformable to the shape of the scalp. Hollow tubing is configured to receive pumped air or gaseous fluid as part of a thermal regulation system. The tubing is arranged in a series of loops or similar configuration such that fluid of any type can be pumped through the system while providing a large contact area to the scalp for the purpose of imparting a temperature change or to maintain a temperature on the scalp surface. GB 2417423 describes a cooling and pressure application device to relieve migraine. The device is made of tubing which is wound around the scalp into a hemispherical shape. WO 03/047479 describes a cap used to cover a scalp cooling device which may act as a mechanism to attach the scalp cooling device to the wearer. US 21019/262223 describes a therapeutic vibration device which can use a flexible bladder. In this case, the bladder is used to secure the device to the head of the user. There is always a vibration element between the wearer and the bladder, so the bladder makes no direct contact for therapeutic effect. Compression is not being used for any therapeutic effect here, merely its function is to secure the device in place. WO 96/10983 describes a scalp blood irrigation device which uses pressure to increase blood flow to promote hair growth and combat androgenic alopecia, commonly known as pattern baldness.

There are several issues with current treatments. Scalp hypothermia technologies are very uncomfortable for the patient. In clinical data published by the manufacturers of commercially available scalp cooling systems, 42% of patients complained of headaches, with 74% reporting scalp pain induced by the cooling. The appetite for a more tolerable treatment is a key driver amongst all relevant stakeholders for a disruptive solution to this need. Treatment centres suffer an opportunity cost due to the length of extra treatment time required—the availability of time slots for scalp cooling treatment can become a limiting factor in the number of additional chemotherapy patients a clinic can treat.

Tourniquet systems modelled on surgical tourniquets to apply pressure to the scalp were investigated in the 1970s and 1980s before being discontinued on the grounds of obsolescence, poor efficacy and potential discomfort and danger to the patient, as the system applied a single point of pressure at a very high level.

Infection transfer also poses a further problem. Scalp cooling caps wrap around the chin area of patients, meaning that bodily fluids left on the non-washable caps risk poorer health outcomes for immunodeficient patients. This also causes ongoing maintenance issues for clinical staff. The efficacy and reliability of current solutions is questioned by stakeholders, with issues such as poor cap fitting resulting in abandoned or failed treatments. As such, there is no widely available solution for CIA which is both comfortable and minimises time in clinic.

CIPN is a widely-reported side effect of many chemotherapy treatments that results in loss of sensory and motor control of the peripheries in addition to chronic pain. CIPN results in varying levels of disability for the patient and can result in cessation or dose reduction of the chemotherapy treatment plan, which can increase morbidity and mortality of the cancer. CIPN is reported in as many as 68% of chemotherapy patients. The additional healthcare system cost burden of patients who develop CIPN is estimated at over $17,000 per patient. The mechanism of CIPN development is not clearly understood but is known that the cellular toxicities caused by frequently used chemotherapy drugs such as paclitaxel lead to the injury of long myelinated fibres in peripheral pathways, resulting in demyelination.

Solutions to prevent CIPN are not in widespread clinical use, but several are documented in literature, including the use of hypothermic techniques like that used in CIA prevention (such as WO2014120090A1) and the use of compositions or other pharmaceutical approaches that attempt to prevent or reverse CIPN by means of a chemical process. However, presently there is no solution widely available to patients or clinicians to prevent the development of CIPN.

CII is caused by chemotherapy drugs interacting with both male and female reproductive systems, leading to premature ovarian insufficiency and infertility in female patients, and the reduction or cessation of sperm production in males. While the exact overall prevalence of CII is unknown, the risk of fertility related side effects of chemotherapy is a consideration in cancer treatments in all men and women who wish to have children after the completion of their chemotherapy treatment. In some cases, chemotherapy induced infertility can be irreversible, leading to higher levels of psychosocial stress and mental health disorders. This global population of patients who are considered to be at risk of CII is estimated to be approximately 1 million patients annually.

There are no widely used or well-documented solutions to prevent CII in male patients. In female patients, several clinical trials have analysed the impact of gonadotropin-releasing hormone agonists (GnRHa) in preserving the ovarian function of breast cancer patients. While the method has shown success in slowing ovarian function (intended to reduce the risk and severity of chemotherapeutic damage), any overall benefit to fertility has not been clinically proven. In addition, the possibility for effectiveness of this approach for other cancers is not clear. In clinical practice, existing solutions for male patients focus on sperm storage, while female patients can pursue ovarian tissue cryopreservation via a surgical procedure (applicable to paediatric and young adult patients only), in-vitro fertilisation or intrauterine insemination. In both cases, current treatment options are highly expensive, with 64% of male patients reporting spend of greater than or equal to $15,000, while female patients can expect costs ranging between $7,000 and $30,000 per patient.

It is an object of the present invention to overcome at least one of the above-mentioned problems.

### SUMMARY OF THE INVENTION

Chemotherapy induced side effects in disease-free tissue are caused by the unintended destruction of fast-dividing healthy cells by chemotherapy drugs. For example, CIA is caused by the unintended destruction of fast-dividing hair cells by chemotherapy drugs. The exact pharmacological mechanism of this action is not yet well understood. As such, most efforts to prevent hair loss during chemotherapy have revolved around preventing or significantly reducing drug delivery and/or drug uptake by the hair follicles.

In one aspect, there is provided a pneumatic pressure device and method to prevent chemotherapy induced side effects by localised, upstream, or both, vascular compression therapy, thus reducing drug delivery and/or drug uptake by the hair follicles.

The method of the claimed invention (localised microvascular compression therapy) involves the application of a small amount of pressure (between about 10 mmHg to about 200 mmHg, preferably between about 15 mmHg and about 190 mmHg, more preferably between about 20 mmHg and about 180 mmHg, optionally between about 20 mmHg and about 150 mmHg, and between about 20 mmHg and about 100 mmHg; ideally between about 25 mmHg to about 75 mmHg; and in one aspect, between about 40 mmHg to about 60 mmHg) across a tissue surface above the target microvasculature, causing the local capillaries in that area to collapse, and the blood in the local vessels to be pushed out. The pneumatic pressure device and method described herein uses this effect to inhibit drug delivery to target tissue by attaching a pneumatic pressure device to the target area of the subject and which applies a constant pressure across the surface of the target tissue.

In one aspect, the invention described herein relates to a pneumatic pressure or compression device. The device of the claimed invention (for example, a pneumatic pressure cap or device) applies a small amount of pressure (between about 20 mmHg to about 100 mmHg, ideally between about 25 mmHg to about 75 mmHg; and in one aspect, between about 40 mmHg to about 60 mmHg) across a surface above a bony prominence, causing the local capillaries in that area to collapse, and the blood in the local vessels to be pushed out. The device described herein uses this effect to inhibit drug delivery to, for example, the hair follicles by attaching the pneumatic pressure device to the head of the subject and which applies a constant and/or uniform pressure across the surface of the scalp. The device described herein also uses this effect to inhibit drug delivery to the outer extremities such as the fingers and toes, and areas such as the vaginal cavity.

There is provided a pneumatic compression device for use in preventing or treating chemotherapy-induced alopecia in a subject receiving chemotherapy treatment, the device (1) comprising a headband member (2), a first layer (3), a second layer (4), an outer mesh layer (6), at least one primary bladder (5), and a control element (9), wherein the at least one primary bladder (5) is located between the first layer (3) and second layer (4), and is configured to inflate with air or a gas at ambient temperature, and which exerts a compressive pressure to the scalp of the subject when the bladder (5) is inflated.

There is provided, in one aspect, a pneumatic pressure device (1) for use in preventing or treating chemotherapy-induced alopecia in a subject receiving chemotherapy treatment, the device (1) comprising a headband member (2) adapted for fitting the device (1) to the head of the subject; the headband member (2) is connected to a first layer (3), a second layer (4), and at least one primary bladder (5) sandwiched therebetween; the at least one primary bladder (5) is attached along its inner edge (5a) to the second layer (4), and along its perimeter surface (5b) to the first layer (3) and along its lower edge surface (5c) to the headband member (2); wherein the first layer (3) is enveloped with an outer mesh layer (6) attached to the headband member (2); and, wherein the at least one primary bladder (5) is configured to inflate with air or a gas at ambient temperature, and which exerts a compressive pressure to the scalp of the subject when the bladder (5) is inflated.

In one aspect, there is provided a pneumatic compression device (1,100) for use in preventing or treating chemotherapy-induced alopecia or chemotherapy-induced peripheral neuropathy (CIPN) in a subject receiving chemotherapy treatment, the device (1,100) comprising: an attachment member (2), a first layer (3) connected to the attachment member (2), at least one primary bladder (5); and a fluid inlet (12), wherein the at least one primary bladder (5) is configured to inflate with a fluid at ambient temperature, and which exerts a compressive pressure to the non-treated area of the subject when the bladder (5) is inflated minimising blood perfusion to prevent chemotherapy delivery to the non-treated area.

In one aspect, the pneumatic compression device (1,100) further comprises a control element (9).

There is provided a pneumatic compression device (100) for use in preventing or treating chemotherapy-induced peripheral neuropathy (CIPN) in a hand or a foot of a subject receiving chemotherapy treatment, the device (100) comprising an attachment member (2), a first layer (3), at least one primary bladder (5), and a control element (9), wherein the at least one primary bladder (5) is located between the first layer (3) and the hand or foot of the subject, and is configured to inflate with air or a gas at ambient temperature, and which exerts a compressive pressure to the hand or the foot of the subject when the bladder (5) is inflated.

In one aspect, the at least one primary bladder (5) is configured to have a plurality of sockets (108) to accommodate the fingers of the hand or the toes of the foot of the subject.

In one aspect, the at least one primary bladder (5) is configured to be a single socket that accommodates the whole of the hand or the foot of the subject.

In one aspect, the pneumatic compression device (100) further comprises a first securing member (102) to secure the device (100) in place.

In one aspect, the pneumatic compression device (100) further comprises a second securing member (103) to secure the device (100) in place.

There is provided a pneumatic compression device (200) for use in preventing or treating chemotherapy-induced infertility (CII) in a subject receiving chemotherapy treatment, the device (200) comprising at least one primary bladder (205), an applicator (202) adapted to accommodate the at least one primary bladder (205), and a cap (204) configured to contain the at least one primary bladder (205) within the applicator (202) when deflated, wherein the at least one primary bladder (205) located within the applicator (202) is configured to inflate with air or a gas at ambient temperature, and which exerts a compressive pressure when the bladder (205) is inflated.

In one aspect, the applicator (202) comprises an outer shell (210) and an inner shell (212), which combine to form an enclosure (214) that accommodates the at least one primary bladder (205).

In one aspect, the pneumatic compression device (200) further comprises a support means (208) housed internally in the applicator (202) and in communication with the cap (204). When the at least one primary bladder inflates, the cap is released from the applicator, and is pushed upwards while being supported by the support means. The support means bisects the at least one primary bladder, which envelopes the support means as it is inflated.

In one aspect, the cap (204) is reversibly connected to the applicator (202) and is released from the applicator (202) when the at least one primary bladder (205) is inflated.

In one aspect, the cap (204) is fixed to the applicator (202) and further comprises an aperture (206) through which the at least one primary bladder (205) is pushed through prior to inflation.

In one aspect, the control element or control system comprises a pump, at least one air vent (12) for accessing air or gas inflow and for allowing for the air or the gas to exit the device, and a tactile on/off switch (14).

In one aspect, the at least one primary bladder (5) further comprises a plurality of bladder compartments, each bladder compartment operating independently of each other.

In one aspect, the at least one primary bladder (5) further comprises a plurality of bladder compartments, each of the plurality of bladder compartments are in fluid communication with a bladder compartment juxtaposed it.

In one aspect, the at least one primary bladder (5) or the plurality of bladder compartments are connected to the pump, the pump having a two-way valve system to control the inflow and outflow of the air or the gas.

In one aspect, the device (1,100,200) further comprises a pressure sensor. Preferably, the pressure sensor is in communication with the at least one primary bladder (5).

In one aspect, the device (1,100,200) further comprises a tissue perfusion sensor.

In one aspect, the device (1) further comprises an outer mesh layer (6) surrounding the second layer (4).

In one aspect, the device (1) further comprises an inner membrane (7), the inner membrane (7) forms an inner lining of the device (1), nestling between the second layer (4) and the scalp of the subject.

In one aspect, the device (1) further comprises a secondary bladder (11), wherein the secondary bladder (11) is configured to attach to the attachment member (2) such that the secondary bladder (11) comes in contact with the head of the subject.

In one aspect, the device (1) further comprises a first fastening system (16) configured to prevent the outward movement of the outer mesh layer (6).

In one aspect, the device (1) further comprises a second fastening system (17) adapted to tighten the attachment member (2) to the head of the subject.

In one aspect, when activated, the device (1,100,200) applies a compressive pressure of between about 30 mmHg to about 350 mmHg, or to about 200 mmHg, to the scalp of the subject. Preferably, the compression pressure is between about 30 mmHg to about 150 mmHg or between about 20 mmHg and 100 mmHg; ideally between about 25 mmHg to about 75 mmHg; and in one aspect, between about 40 mmHg to about 60 mmHg.

In one aspect, the compressive pressure is applied incrementally.

In one aspect, the compressive pressure is applied uniformly or non-uniformly.

In one aspect, the attachment member (2) further comprises at least one status indicator light (13).

In one aspect, the device (1) further comprises at least one adjustable strap (10) to secure the device under, around or over the chin of the subject.

In one aspect, there is provided a method for preventing or treating chemotherapy-induced damage to an off-site area of the body of a patient, the method comprising incrementally applying a compressive pressure to the off-site area (for example, the pneumatic compression device is in situ at the target site; the target site can either be the protected tissue site (in the case of the scalp) or a vessel structure upstream of the protected tissue (for example, targeting the uterine artery that supplies the female reproductive organs via the vaginal cavity); and modulating the applied pressure to balance unwanted drug delivery and the avoidance of hypoxia damage to tissue at the occlusion and protected site.

When the pneumatic compression device incrementally applies the compressive pressure to the occlusion site, the occlusion site should begin to become partially occluded as the compressive system pressure increases incrementally in an electronically or mechanically controlled manner. The pressure applied should reach a peak of between 20 mmHg and about 200 mmHg (preferably between about 20 mmHg and about 100 mmHg; ideally between about 25 mmHg to about 75 mmHg; and in one aspect, between about 40 mmHg to about 60 mmHg) depending on the site of application, such that the user is comfortable, maintaining this pressure once a specific threshold is reached (40-60 mmHg) where the localised blood flow is reduced by at least 60%. The pressure is maintained in a uniform distribution at each point at the occlusion site for a specified period of time relating to the half-life of the drug being used to treat the patient, this time period controlled electronically or mechanically by the control system. As the pressure reaches a peak, the local blood vessels and microvasculature becomes almost completely occluded at the occlusion site. This occlusion reduces or avoids drug delivery through the vasculature to the protected site, ultimately preventing side effects at the protected site. Once the specified time period of applying the pressure has elapsed, the pressure is decreased slowly in a manner controlled electronically or mechanically by the control system so as to avoid sudden reperfusion of the tissue.

The pneumatic compression device will operate for a pre-determined time period based on the half-life of the drug intervention being used.

At the expiration time, the pneumatic compression device begins decrementing the pressure applied to allow safe incremental increases in blood perfusion in the effect site avoiding reperfusion injury at both the occlusion and protected site.

At the expiration time, the pneumatic compression device begins to decrease the applied pressure in a pulsatile fashion, whereby the pressure is reduced significantly for a period of 5 seconds, then re-applied for a period of 5 seconds, followed by a further brief period of pressure reduction and so on. In this aspect, safe increments of blood perfusion in the effected site are allowed while also increasing blood flow to drive toxins away from the site.

In one aspect, the reduction in perfusion at the target site and the site of application is monitored by sensing one of the following parameters: SpO2, red blood cell count, haemoglobin concentration.

In one aspect, the compressive force is modified based on the sensed parameter such that the ideal amount of compressive force is applied independently of the user.

In one aspect, the device further comprises a foam layer inserted within the bladder or one or more of the plurality of bladders. The foam is a low-density polymer material such as low-density polyurethane. In this aspect, the pump connected to the air inlet is connected in reverse polarity, causing air to be withdrawn from the bladder. The pump withdraws fluid (and hence, pressure) incrementally, creating a vacuum inside the bladder. This causes the outer first layer and the bladder to collapse on the foam layer, causing the foam layer to compress. When in a compressed state, the foam layer exerts a reaction force through the bladder onto the scalp, foot, hand, or within a cavity, creating the compression levels necessary to constrict blood flow.

In one aspect, the fastening systems or adjustment member can be used with greater force to tighten the device against the surface of the scalp, foot, or hand. Through a drawstring mechanism of tightening the device against the scalp/foot/hand surface, pressure levels required for therapeutic effect are achieved.

In one aspect, a fluid or solid which takes a viscous form at ambient pressure, but a solid form at pressures lower than atmospheric pressure, is inserted into the bladder or one or more of the plurality of bladders. In this aspect, the pump connected to the air inlet is connected in reverse polarity, causing air to be withdrawn from the bladder, and forming a vacuum inside the bladder. As the pressure within the bladder decreases, the substrate hardens, creating a modifiable compressive force against the tissue (scalp, foot, hand or within a cavity).

In one aspect, the bladder or the plurality of bladders inflated are inflated using an air inlet to a pre-determined pressure level below the target therapeutic pressure level. Using the fastening systems or adjustment member (such as a drawstring or hook-and-eye strapping), compressive pressure is applied to the bladder, reducing the volume of the bladder in a modifiable way. This reduction in volume of the bladder causes an increased internal pressure in the bladder, causing modifiable pressure levels as the target therapeutic range to be applied to the tissue (scalp, foot, hand or within a cavity).

In one aspect, the bladder or the plurality of bladders are provided at a pre-determined pressure level and is not modified by an air inlet.

In one aspect, there is provided a method for preventing or treating chemotherapy-induced alopecia in a subject receiving chemotherapy treatment, the method comprising the steps of:
fitting the pneumatic compression device (1) described above to the subject; and
activating the switch (14) at the rear of the attachment member (2) to initiate inflation of the at least one primary bladder (5) for a specified time.

In one aspect, when the switch (14) is activated, the device inflates and applies incremental pressure against the scalp of the user, as described above. The device maintains the pressure uniformly across the scalp surface for a pre-set period of time. When this time period is complete, the device deflates the bladder in the device in a controlled and decremental manner that is predetermined in advance, which avoids any reperfusion injury to the user.

In one aspect, the method further comprises tightening the device (1) to the scalp of the subject by activating the first fastening system 16.

In one aspect, the method further comprises tightening the pneumatic compression device (1) to the scalp of the subject by activating the second fastening system 17.

In one aspect, the method further comprises securing the adjustable strap (10) under the chin of the subject.

In one aspect, when the period of time is completed and the bladder is deflated, the user may remove the device by loosening the fastening systems.

In one aspect, there is provided a method for preventing or treating chemotherapy-induced peripheral neuropathy in a subject receiving chemotherapy treatment, the method comprising the steps of:
fitting the pneumatic compression device (100) described above to the subject; and
activating a switch on the control element to initiate inflation of the at least one primary bladder 5 for a specified time.

In one aspect, the method further comprises tightening the device (100) to the foot or hand of the subject by activating the first securing means or the second securing means, or a combination of both.

In one aspect, there is provided a method for preventing or treating chemotherapy-induced infertility in a subject receiving chemotherapy treatment, the method comprising the steps of:
fitting the pneumatic compression device (200) described above to the subject; and
activating a switch on the control element to initiate inflation of the at least one primary bladder (205) for a specified time.

In one aspect, there is provided a method of controlling drug delivery via the blood vessels to a target site on the body, the method comprising:
mechanically compressing the skin/surface tissue at a target tissue site at a predetermined compressive force to occlude the upstream blood vessels;
maintaining the compressive force for a predetermined period (such that drugs circulating through the vascular system do not reach the target site); and releasing the compressive force applied to the tissue at the target site at a pre-determined rate and in a pre-determined geometric pattern (releasing the compressive force comprises lessening the mechanical compression force such that long-term tissue damage and ischemia reperfusion injury may be avoided).

When the vessels are occluded, this causes occlusion of the local microvasculature and superficial arteries with limited possibility for drug delivery at the target tissue site or at an organ site downstream of the target tissue site.

In one aspect, when the compressive force is applied to the tissue site upstream of the intended effect site. This means that the compressive pressure is applied to target vessels at a different point to the site of the intended protective effect.

In one aspect, the compressive force is applied to the skin/surface tissue at the target site at about 20 mmHg to about 100 mmHg.

In one aspect, the compressive force applied is configured to mechanically occlude the microvasculature close to the surface of the tissue and local arterial supply vessels.

In one aspect, the compressive pressure is applied uniformly across the tissue site to be treated.

In one aspect, the compressive force is maintained for a period of between 30 minutes and 7 hours.

In one aspect, the compressive force is applied at different areas of the tissue such that larger vessels can be met with larger levels of compression.

In one aspect, the compressive force is applied in a pulsatile fashion.

In one aspect, there is provided a method of controlling drug delivery to a target site on the body, followed by a period of reactivating blood flow to ensure long-term tissue viability, the method, comprising:
mechanically compressing the skin/surface tissue at the target site with a predetermined compressive force to cause occlusion of the local microvasculature with limited possibility for drug delivery;
maintaining microvasculature occlusion for a predetermined period of time such that drugs circulating through the vascular system do not reach the target site;
releasing the occlusion in a controlled manner at a pre-determined rate and in a pre-determined geometric pattern such that long-term tissue damage and ischemia reperfusion injury may be avoided;
sensing a physiological parameter;
adjusting the mechanical compression applied to the tissue site based on the sensed physiological parameter; and
applying compressive forces in a pulsatile fashion against the skin tissue to promote blood flow allowing waste agents to be removed from the local vasculature.

In one aspect, the sensed physiological parameter is the blood perfusion in the tissue site being treated. The blood perfusion in the tissue is typically measured by magnetic resonance imaging (MRI), positron emission tomography (PET) and laser doppler methods. MRI is a non-invasive technique to directly measure blood flow by utilizing arterial blood as an endogenous tracer. PET scans require the introduction of a radioactive tracer into the blood supply. Laser Doppler flowmetry (LDF) is an established surface technique for the real-time measurement of red blood cell motion at the surface of tissue. LDF and laser Doppler imaging (LDI) work by illuminating the tissue with a laser. The perfusion measurement is derived from the product of the mean velocity and the concentration of the red blood cells within the volume of the tissue being measured. Perfusion is measured at the surface of the tissue and is only given as relative values.

### Definitions

In the specification, the term “healthy” should be understood to mean where the individual or patient has no underlying medical condition, infection, inflammatory response, condition or otherwise occurring.

In the specification, the term “cancer” should be understood to mean a cancer selected from the group comprising node-negative, ER-positive breast cancer; early stage, node positive breast cancer; multiple myeloma, prostate cancer, glioblastoma, lymphoma, fibrosarcoma; myxosarcoma; liposarcoma; chondrosarcoma; osteogenic sarcoma; chordoma; angiosarcoma; endotheliosarcoma; lymphangiosarcoma; lymphangioendotheliosarcoma; synovioma; mesothelioma; Ewing's tumour; leiomyosarcoma; rhabdomyosarcoma; colon carcinoma; pancreatic cancer; breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumour; cervical cancer; uterine cancer; testicular tumour; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukaemia's. Also included are metastases selected from the group comprising: bone metastases; lung metastases; liver metastases; bone marrow metastases; breast metastases; and brain metastases.

In the specification, the term “individual”, “subject”, or “patient” should be understood to mean an animal, which incorporates all mammals, for example, a human, primates, non-human primates, farm animals (such as pigs, horses, goats, sheep, cows (including bulls, bullocks, heifers etc.), donkey, reindeer, etc.), veterinary mammals (such as dogs, cats, rabbits, hamsters, guinea pigs, mice, rats, ferrets, etc.), and mammals kept in captivity (such as lions, tigers, elephants, zebras, giraffes, pandas, rhino, hippopotamus, etc.), and other mammals and higher mammals for which the use of the invention is practicable.

In the specification, the term “treatment” should be understood to mean prohibiting, preventing, restraining, and slowing, stopping or reversing progression or severity of, for example, chemotherapy-induced alopecia (CIA), chemotherapy-induced peripheral neuropathy (CIPN), and chemotherapy-induced infertility (CII).

In the specification, the term “vasoconstriction” should be understood to mean the narrowing of the blood vessels resulting from contraction of the muscular wall of the vessels, in particular the large arteries and small arterioles, reducing blood flow in those blood vessels to reduce the delivery of chemotherapeutic agents, for example, to the scalp to limit hair loss. The main arteries of the scalp are the supratrochlear, supraorbital, superficial temporal and occipital; while the major veins are the superficial temporal, posterior auricular and occipital. The main arteries of the extremities are the radial, ulnar, brachial, dorsalis pedis, anterior tibial, lateral calcaneal, medial calcaneal, lateral plantar, and medial plantar. The main arteries of the pelvic cavity are the superior vaginal arteries, uterine arteries and ovarian arteries.

In the specification, the term “bladder” should be understood to mean, in the context of the device of the claimed invention, a hermetically sealed pouch that can be filled with a fluid and which is configured to withstand pressures of up to 500 mmHg.

In the specification, the term “fluid” should be understood to mean a substance that has no fixed shape, flows easily, and yields easily to external pressure. Examples of a fluid include a gas, air, or a liquid.

In the specification, the term “shell” should be understood to mean a carapace or protective casing, which can be made of hard, soft, or compliant (malleable) material. Examples of hard material include hard plastic, metal (such as aluminium, stainless steel) fibreglass, carbon fibre, graphene, acrylonitrile butadiene styrene (ABS), polylactic acid (PLA), and similar materials. Examples of soft material include cotton, linen, silk, foam, polyurethane (PU) Gel/foam, silicone, latex, polyvinyl chloride (PVC), polyethylene (PE), polyester, bamboo fabric, and the like. Examples of compliant material include rubber, memory foam, and the like. Other components that could be embedded with the soft or compliant materials mentioned above include antibacterial agents such as silver, copper, zinc, and the like.

In the specification, the term “pressure sensor” should be understood to mean a device which measures the fluid pressure inside a bladder. Typically, the sensor comprises of a resistive, capacitive, or inductive sensing element, and can measure pressures from 0 to about 100 kPa; preferably from about 0 to about 80 kPa; more preferably from about 0 to about 50 or about 60 kPa; and ideally from about 0 to about 40 kPa.

In the specification, the term “tissue perfusion sensor” should be understood to mean a device which measures the level of blood perfusion in tissue, for example the scalp tissue. Typically, the sensor comprises a non-invasive convective blood perfusion probe which adopts to the shape of the area being treated. For example, for the scalp compression, the sensor is circular in shape and sits on the scalp surface. The perfusion measurement is derived from the product of the mean velocity and the concentration of the red blood cells within the volume of the tissue being measured. The measurement range is typically about 1, 2, 3, 4, or 5 mm in tissue depth below the probe.

In the specification, the term “pneumatic” should be understood to mean the use of a gas or air operated under pressure.

In the specification, the term “ambient temperature” should be understood to mean the average air temperature surrounding something (such as a person). Typically, it means “room temperature”, which means a dry, clean, well-ventilated area at temperatures between 15° to 25° C. (59°−77° F.) or up to 30° C., depending on climatic conditions.

In the specification, the term “cavity” should be understood to mean a nasal cavity, a buccal cavity, and the pelvic cavity (including the reproductive organs, the vaginal cavity, urinary bladder, distal ureters, proximal urethra, terminal sigmoid colon, rectum, and anal canal. In the female, the uterus, Fallopian tubes, ovaries and (upper) vagina occupy the area between the other viscera).

In the specification, the term “non-treated area” should be understood to mean a non-targeted area of the body that is not afflicted by the cancer being treated.

In the specification, the term “control element” and “control system” are interchangeable and should be understood to typically contain a pump, at least one air or a gas vent for accessing air or gas inflow and for allowing air or gas to flow back out, at least one pressure sensor and a tactile on/off switch. The at least one pressure sensor can also be located within the at least one primary bladder or on the tissue-facing side of the at least one primary bladder and remain connected to the control element/system. This control element or system further comprises a controlling electronic architecture for reading inputs from the pump and the at least one air pressure sensor. The pump is powered by a battery which is contained within the control element/system.

### BRIEF DESCRIPTION OF THE DRAWINGS

The invention will be more clearly understood from the following description of an embodiment thereof, given by way of example only, with reference to the accompanying drawings, in which:—

FIG. 1 illustrates a perspective view of a pneumatic compression device of the claimed invention.

FIG. 2 illustrates the pneumatic compression device of FIG. 1 with a cut-away portion showing the layers of the device.

FIG. 3A illustrates a control element of the claimed invention for use with the pneumatic compression device of FIG. 1, while FIG. 3B illustrates the control element affixed to the rear of the device of the claimed invention. The scalp compression device of FIG. 1 in use, where (a) the device is placed on the head of the patient, (b) a dial at the back of the device is turned to tighten and align the device on the head of the patient, and (c) pressure sensors activate a motorised dial at the front of the device to gradually apply and control the pressure in the device to create the desired compression.

FIG. 4 illustrates a rear perspective view of the device illustrated above with a secondary bladder indicated by the dotted line.

FIG. 5 illustrates a device of the claimed invention for use on the hand of a subject receiving chemotherapy treatment.

FIG. 6 illustrates the device of FIG. 5 with the outer layer in cross section, revealing the primary bladder beneath.

FIG. 7 illustrates the device of FIG. 5 and FIG. 6 in cross section.

FIG. 8 illustrates the device of the claimed invention for use on the foot of a subject receiving chemotherapy treatment.

FIG. 9 illustrates the device of FIG. 8 with the outer layer in cross section, revealing the primary bladder beneath.

FIG. 10 illustrates a device of the claimed invention for use in a cavity of a subject receiving chemotherapy treatment, where (A) shows the device in situ but not deployed; (B) shows the device during deployment; and (C) shows the device fully deployed, while (D) shows the fully deployed device in (C) in cross-section, revealing the inner elements of the device.

FIG. 11 illustrates a device of the claimed invention for use in a cavity of a subject receiving chemotherapy treatment, where (A) shows the device in situ but not deployed; (B) shows the device during deployment; (C) shows the device in (B) in cross-section, revealing the inner elements of the device; and (D) shows the device fully deployed.

FIG. 12 illustrates a graph showing the reduction in tissue perfusion compared to perfusion at baseline at a target site (in the case the scalp) in eight human volunteers when varying levels of compressive pressure are applied using a device of the claimed invention. Tissue perfusion is measured in relative perfusion units as provided by a Moor Instruments Laser Doppler monitor.

### DETAILED DESCRIPTION OF THE DRAWINGS

Localised microvascular compression therapy has applications in the prevention of CIA, CIN, CII, and other drug side effects such as dry skin, and mucositis. The pneumatic compression device of the claimed invention modulates localised drug delivery to protect off-target tissue. The pneumatic compression device applies tissue and/or vessel compression, in a range of 20 mmHg to 200 mmHg (preferably from about 20 mmHg to about 100 mmHg), from a pneumatic compression against unbroken (not punctured) skin or internal structures (such as within the vaginal cavity), to occlude local blood vessels, leading partially or completely to reduced blood perfusion at the protected site, and consequently reduced drug delivery at an effect site (or protected tissue site). This modulation of localised drug delivery reduces or eliminates unwanted side effects at the protected tissue site caused by drugs in the circulatory system.

Firstly, the pneumatic compression device will apply pressure over a large area to reduce blood prefusion to the protected tissue. This large area approach allows for lower pressure levels (20-100 mmHg) compared to traditional tourniquet devices (these apply high pressure over a small region). This large pressure footprint allows for greater patient comfort during treatment. Secondly, the applied pressure is modulated during treatment to reduce or avoid perfusion related damage to the tissue at the occlusion site or the protected site. The reduction of pressure post therapy will also be controlled to avoid reperfusion injury.

The above pneumatic compression device can significantly reduce off-target drug-related damage to skin, peripheral nerve endings, ovaries or bladder, amongst other tissues and organs.

The present invention provides a pneumatic compression device designed to apply either uniform or non-uniform incremental pressure to an area of the subject for the therapeutic purpose of preventing damage done by chemotherapeutic drugs. Before chemotherapy infusion, the device can be fitted to the head, foot, hand or within a cavity of the subject, fastened in place (if appropriate), and then activated. The device may be used with any size or shape of head, foot, hand, or cavity, which allows it to be used for all ages (from an infant to an adult), providing a flexibility and ease of use. An inner bladder mechanism inflates incrementally using a fluid at room temperature to apply (uniform/non-uniform) pressure to the area of interest, for example, the scalp. With this level of therapeutic pressure applied before, during and after chemotherapy treatment, the device causes local vasoconstriction of the blood vessels, reducing the tissue perfusion in the area of interest, and thus preventing drug delivery to, for example, the hair follicle or other non-target areas.

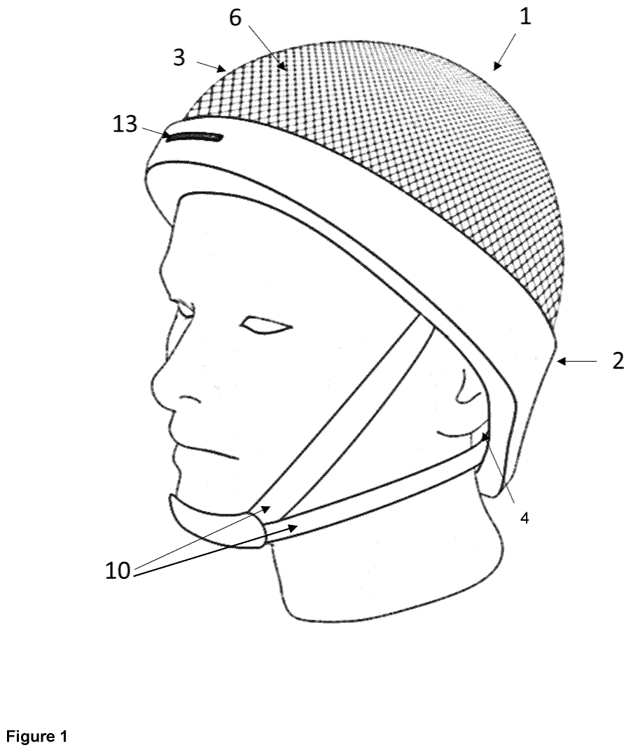

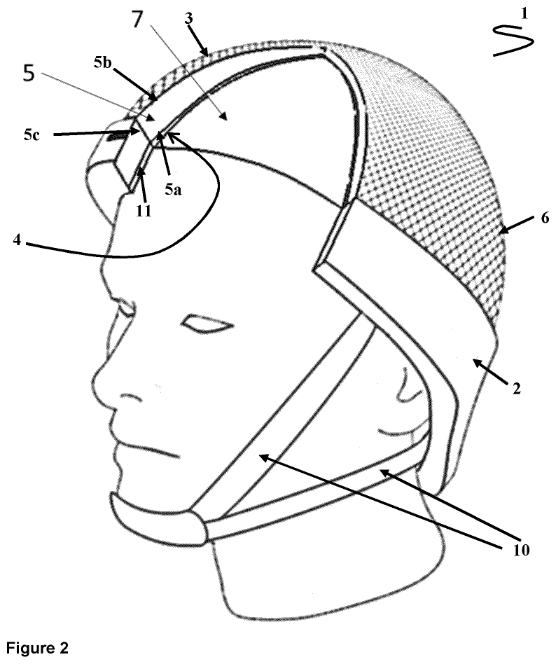

Referring now to the figures, where FIG. 1 illustrates a general embodiment of a pneumatic compression device of the present invention for use with a scalp of a subject. Specifically, FIG. 1 illustrates a perspective view of the pneumatic compression device of the present invention fitted to the head of a subject and is generally referred to by reference numeral 1. The device 1 of the illustrated embodiment comprises an attachment member 2 adapted for fitting the device 1 to the head of the subject. The attachment member 2 is connected to a first layer 3, a second layer 4 (see FIG. 2), and at least one primary bladder 5 sandwiched therebetween (see FIG. 2). The attachment member 2 acts as an anchor for the other elements of the device 1. The at least one primary bladder 5 is attached along its inner edge 5a to the second layer 4, and along its perimeter surface 5b to the first layer 3 and along its lower edge surface 5c the attachment member 2. The at least one primary bladder 5 is inflated using a fluid, preferably air or a gas, which is preferably at room temperature. The first layer 3 is typically encased with an outer mesh layer 6, which is also attached to the attachment member 2. The attachment member 2 is typically rigid.

When inflated, the at least one primary bladder 5 expands and applies pressure to the scalp, as the bladder 5 can only expand towards the scalp. The outer mesh layer 6 stops any outward expansion of the device 1 away from the scalp. One of the functions of the outer mesh layer 6 is to constrain the primary bladder 5 so that air pressure inside the primary bladder 5 is applied to the scalp. If a series of interconnected primary bladders 5 or a series of bladder compartments are used, varying amounts of pressure can be applied to the same or to different areas of the scalp.

The device 1 further comprises an inner membrane 7. The inner membrane 7 ensures that the device 1 is a comfortable fit on the subject. In one aspect, the inner membrane 7 runs in conjunction with the attachment member 2, that is, circumferentially around the head of the subject. The inner membrane 7 forms a seal around the scalp of the subject. In one aspect, the inner membrane 7 forms the innermost lining of the device 1 and nestles between the second layer 4 and the head of the subject, substantially enveloping the entire area of the scalp when the device 1 is fitted to the subject. The inner membrane 7 is typically a soft or malleable (compliant) material that is comfortable for the wearer.

The inflatable at least one primary bladder 5 is configured to connect to a pump which inflates the at least one primary bladder 5 with air or a gas when the device 1 is switched on. The pump, and any related electronic controlling architecture, is contained within a removable control element 9 attached within a slot 20 located in the attachment member 2 at the rear of the device 1. The at least one primary bladder 5, which is constrained in place by both the attachment member 2 and the outer mesh layer 6, is thus enabled to apply an incremental compressive force on the head of the subject wearing the device 1.

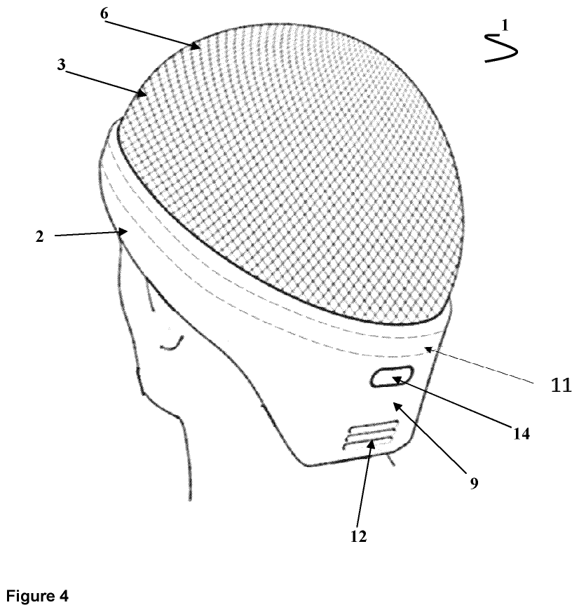

In use, the device 1 is positioned on the scalp of the subject and can be fastened in place by a plurality of adjustable straps 10 affixed to the attachment member 2, which are either anchored on, around or under the chin of the subject (see FIG. 2). In one aspect, adjustable straps can be replaced with a drawstring system attached to the sides of the device 1. In one aspect, the device 1 is secured on the scalp by a secondary bladder 11 which is located on the inner surface of the attachment member 2 such that it is in contact with the forehead, temples and nape of the subject wearing the device 1 (see FIG. 1, FIG. 4). When fluid, preferably air or a gas, is provided to the secondary bladder 11, it inflates to create a seal against the forehead, temples and base or nape of the head of the subject, securing the device 1 in place and creating a seal while the at least one primary bladder 5 is filled with air or a gas and applies the therapeutic compression to the scalp.

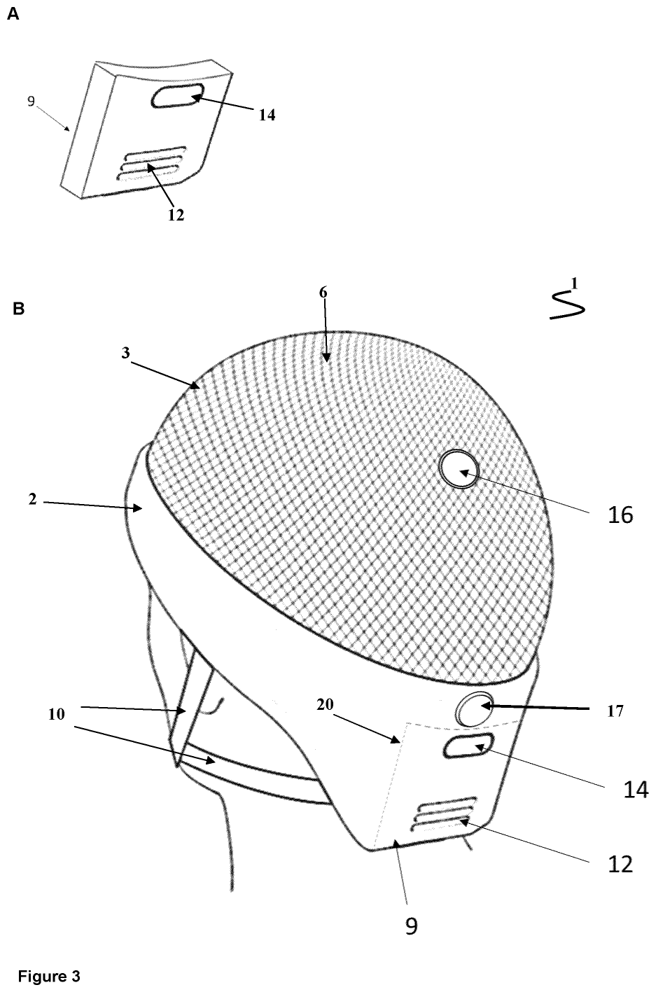

Turning now to FIG. 3a and FIG. 3B, the control element 9 is shown isolated (FIG. 3A) and in situ within the slot 20 (FIG. 3B). The control element 9 typically contains the pump, at least one air or a gas vent 12 for accessing air or gas inflow and for allowing air or gas to flow back out, at least one pressure sensor and a tactile on/off switch 14. The at least one pressure sensor can also be located within the at least one primary bladder 5 or on the scalp-facing side of the at least one primary bladder 5 and remain connected to the control element 9. This control element 9 further comprises a controlling electronic architecture for reading inputs from the pump and the at least one air pressure sensor. The pump is powered by a battery which is contained within the control element 9 of the device 1. The battery can be a rechargeable battery or a single use battery.

The headband member 2 also incorporates at least one status indicator light 13 (see FIG. 1), that is in communication with the control element 9. The status indicator light 13 communicates to the control element 9 via a wired connection through the attachment member 2 and into the control element 9. The at least one indicator light 13 is used to indicate whether the device 1 is on and functioning, turned off, or malfunctioning.

Any incremental changes in pressure is measured by the at least one air pressure sensor, which is connected to the at least one primary bladder 5. The compressive pressure that is applied to the scalp by the device 1 is in the range of about 20 mmHg to about 350 mmHg, or to about 200 mmHg; but preferably between about 20 mmHg to about 150 mmHg, more preferably between about 20 mmHg and about 100 mmHg; ideally between about 25 mmHg and about 75 mmHg. Typically, the compressive pressure applied is between about 40 mmHg and 60 mmHg. When activated, the pump contained within the device 1 inflates the at least one primary bladder 5 to a specified compressive pressure, thus acting like a pneumatic device. This compressive pressure causes local vasoconstriction and a reduced level of skin tissue perfusion, which prevents drug delivery to the hair follicle. When chemotherapy treatment is completed, the device 1 is deflated using a valve system or using the pump in reverse polarity.

In one aspect, the device 1 comprises several air pressure sensors located at several points within the device 1. When the pressure reaches a certain threshold, either low (for example, 30 mmHg or 40 mmHg pressure) or high (for example, between about 60 mmHg to about 200 mmHg pressure), at one or more of the points measured by the pressure sensors, the pump is activated to either pump more air (fluid) into the at least one primary bladder 5 causing more scalp compression, or removing air from the at least one primary bladder 5 causing less scalp compression, as required. In one aspect, the at least one primary bladder 5 comprises several individual bladder compartments, each bladder compartment corresponding to a different section of the device 1 and a different part of the scalp of the subject. Each individual bladder compartment is connected to its own pump in the control element 9 and pressure sensor.

In one aspect, a tissue perfusion sensor, such as a laser Doppler blood flow sensor, is incorporated within or on the scalp-facing surface of the second layer 4 or the attachment member 2. The tissue perfusion sensor analyses the level of blood flow in the scalp tissue being compressed. Using an embedded software system and controlled by the architecture of the control element 9, this information is used to activate the pump to either increase or decrease the pressure in the at least one primary bladder 5 or the bladder compartments.

The tissue perfusion sensor can be incorporated in several ways. In one aspect, the tissue perfusion sensor is incorporated in a hollow in the material making up the second layer 4 so that the sensor is in contact with the scalp of the subject. In one aspect there are at least two tissue perfusion sensors incorporated within the attachment member 2 so that the positioning of the tissue perfusion sensors corresponds to the temple area of the user on each side of the head. In another aspect, there are a plurality of tissue perfusion sensors configured to be patterned around the scalp by being adhered to or within the at least one primary bladder 5 in a particular pattern, or within or on each of the polarity of bladder compartments, ensuring that the perfusion sensors are scattered around and cover the scalp area. In a further aspect, a plurality of tissue perfusion sensors are integrated into the inner shell 4 such that they are in direct contact with the scalp of the subject. The use of a single, two or a plurality of tissue perfusion sensor, permits the user to determine blood flow at different points of the scalp of the subject and to determine how much pressure is needed to constrict the blood vessels to prevent blood flow at that point in the scalp.

In one aspect, the sensors (air pressure, tissue perfusion) and pump(s) further comprise a power source, circuitry to measure its resistance and a means to control the sensors and pump(s) and record readings. In other aspects of any embodiment described here, the air pressure sensor, the tissue perfusion sensor and the pump(s) can further comprise a wireless technology for exchanging data between the sensors and pump(s) and the control element 9 of the device 1 over short distances using, for example, short-wavelength UHF radio waves (for example, Bluetooth®), other wireless data transmission methods such as wireless mesh networks targeted at battery-powered devices in wireless control and monitoring applications (such as ZigBee®), local area networking of devices and Internet access (such as WIFI®), and other wireless communications protocol using a mesh network using low-energy radio waves to communicate from appliance to appliance (such as Z-Wave®). Typically, a remote app can be used on or with the device 1 to record the readings from the air pressure sensor, tissue perfusion sensor, and/or the pump(s). This way, the user can control the inflation of the at least one primary bladder 5, the second bladder 11 and the bladder compartments remotely. Alternatively, the device 1 itself controls the inflation pressure based on feedback from the sensors to the control element 9. The sensors (air pressure and tissue perfusion) and the pump can also be connected to the control element 9 physically by wiring.

At the rear of the device 1 there is a first fastening system 16 which is configured to tighten the outer mesh layer 6 in order to fasten the device 1 to the head of the wearer (see FIG. 3B). In use, and to provide an initial fastening of the device 1 to the head of the subject, the subject twists the first fastening system 16 in one direction (for example, in a clockwise direction) to tighten the outer mesh layer 6, and twists the first fastening system 16 in the opposite direction (for example, in an anticlockwise direction) to loosen the outer mesh layer 6.

In a similar fashion, a second fastening system 17 is used to tighten the attachment member 2 to the temples, forehead, and nape of the subject. This means that the subject may place the device 1 in a comfortable position, tighten the headband member 2 in a mechanical fashion initially by twisting the first fastening system 16 and/or the second fastening system 17 to ensure the device 1 is positioned correctly for the subject, and then activate the on/off switch 14 to fill the secondary bladder 11 and create a seal around the forehead, temples and lower regions of the head of the subject to keep the device 1 in place.

The first and second fastening systems 16,17 can be adjusted manually by the user or adjusted electronically by the architecture of the control element 9. The first and second fastening systems 16,17 typically have a motor within their structure which twists the fastening elements of the systems 16,17. It should be noted that the device 1 can be used without the first and second fastening systems 16,17; or used with only one of the first and second fastening systems 16,17 engaged.

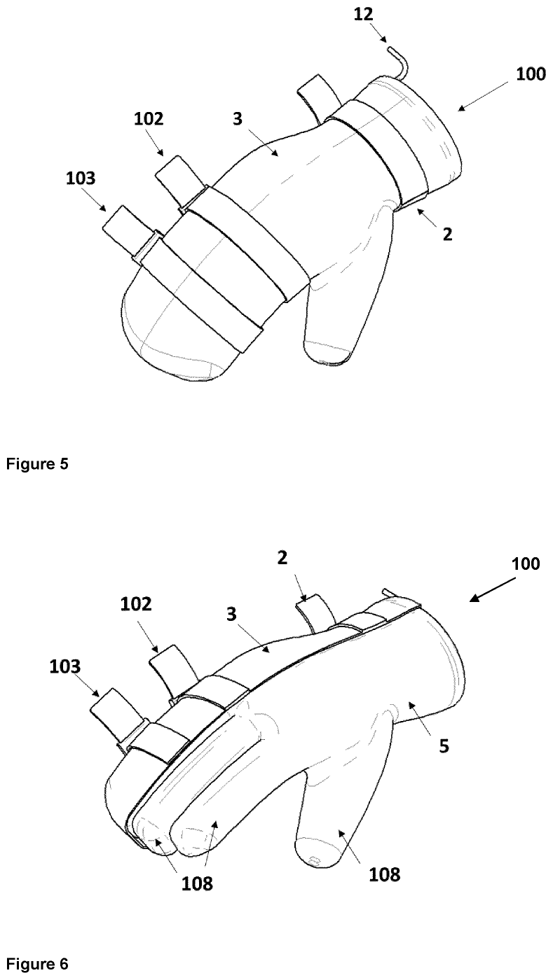

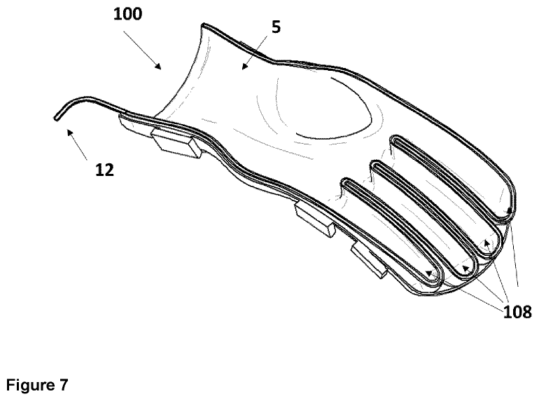

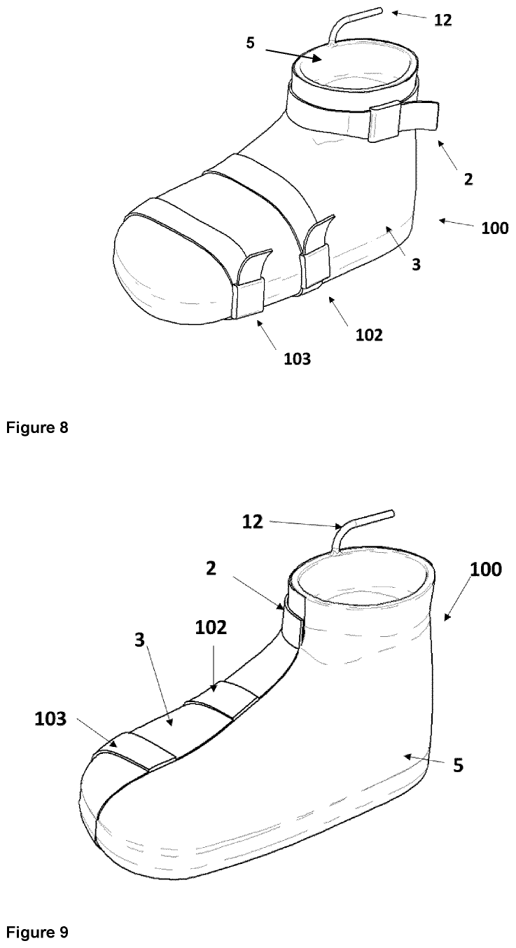

Referring now to the figures, where FIGS. 5 to 9 illustrate a general embodiment of a pneumatic compression device of the present invention for use with a hand or a foot of a subject. Where elements of each embodiment are shared, the same reference numerals are used. Specifically, FIG. 5 illustrates a perspective view of the pneumatic compression device of the present invention fitted to the hand of a subject and is generally referred to by reference numeral 100. The device 100 comprises a first layer 3 in the shape of a glove or mitten which can be fitted over the hand of the user. The first layer 3 is secured to the wearer using an attachment member 2, which tightens the device 100 against the wearer. In addition, a first and a second securing member 102,103, respectively, can also be used to secure the first layer 3 in place around the fingers of the user. At least one primary bladder 5, connected by a single fluid inlet 12, is attached to an inner surface of the first layer 3 (see FIG. 6 and FIG. 7). The at least one primary bladder 5 is arranged such that each point on the skin of the fingers, wrist and forearm of the wearer is in contact with the primary bladder 5. The fluid inlet 12 is then connected to a pump (not shown), electronically coupled with a pressure regulating system/sensor. The pump drives the fluid (such as air, oxygen, water) into the fluid inlet 12 and then into the at least one primary bladder 5. The primary bladder can be arranged to have a plurality of sockets 108, which accommodate the fingers and thumb of the hand of the user (see FIG. 6 and FIG. 7). Alternatively, a network of primary bladders 5 are provided that can form contacts with individual fingers and the thumb of the user. The at least one primary bladder 5 inflates and incrementally applies compressive pressure against the skin of the wearer. The device 100 is programmable to remain inflated until the pressure reduces the skin perfusion of the user and drug delivery is prevented. When a treatment is completed, the device 100 is deflated using the pump and a fluid outlet (or the fluid inlet (12) in reverse).

In one aspect, one or more perfusion sensors are placed on the surface of the plurality of sockets 108 forming the at least one primary bladder 5, which are in contact with the skin of the wearer. The inputs from the perfusion sensors can be used to modulate the level of compressive pressure applied by the at least one bladder 5 to the wearer.

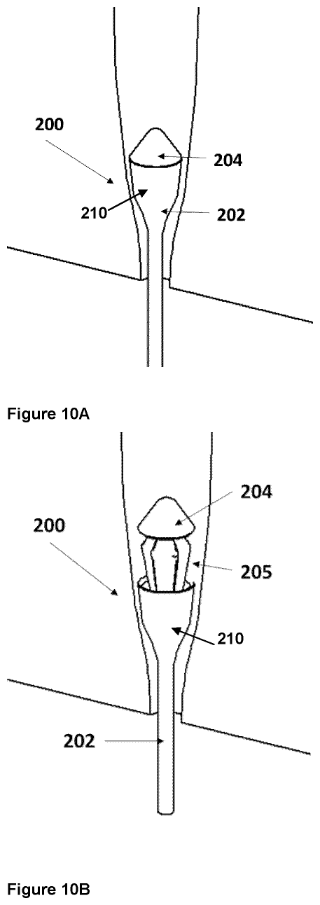

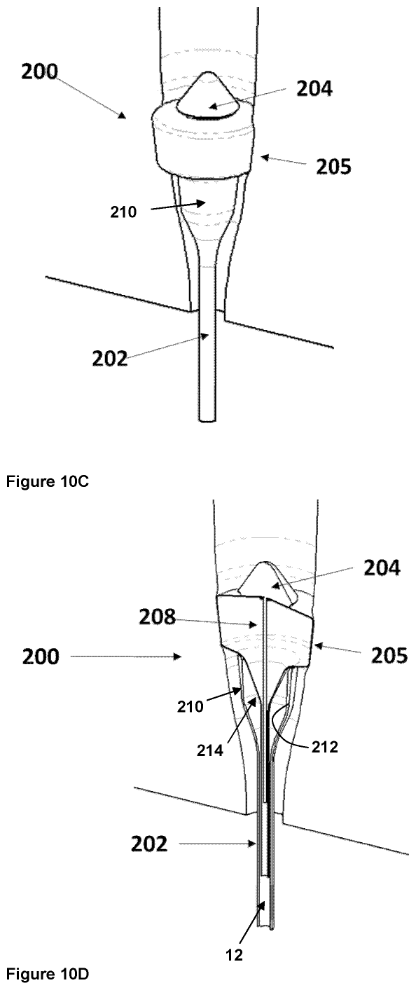

Referring now to FIG. 8, there is illustrated a general embodiment of a pneumatic compression device of the present invention for use with a foot of a subject. Where elements of each embodiment are shared, the same reference numerals are used. Specifically, FIG. 8 illustrates a perspective view of the pneumatic compression device of the present invention fitted to the foot of a subject and is generally referred to by reference numeral 100. The first layer 3 is formed in the shape of a sock which can be fitted over the foot of the user. In this embodiment, the at least one primary bladder 5 is arranged such that each skin surface on the toes, foot, and ankle of the user is in contact with the at least one primary bladder 5. In one aspect, a network of primary bladders 5 are provided that can form contacts with individual toes, as well as the foot and ankle of the user. At least one primary bladder 5, connected by a single fluid inlet 12, is attached to an inner surface of the first layer 3 (see FIG. 9).

A sterile contact layer is placed between the at least one primary bladder 5 and the skin surface of the wearer. This sterile contact layer is typically made from a biocompatible, sterilisable plastic such as polyethylene. The first layer 3 is secured to the wearer using an attachment member 2, which tightens the device 100 against the wearer. Further, a first and second securing member 102,103, respectively, can also be used to secure the first layer 3 in place around the foot of the user. The fluid inlet 12 is then connected to a pump (not shown), electronically coupled with a pressure regulating system/sensor, that is, a control system (as described for the device 1). The pump drives the fluid (such as air, oxygen, water) into the fluid inlet 12 and then into the at least one primary bladder 5. A network of primary bladders 5 are provided that can form contacts with individual toes, the heel, ball of the foot, and ankle of the user. The at least one primary bladder 5 inflates and incrementally applies compressive pressure against the skin of the wearer. The device 100 is programmable to remain inflated until the pressure reduces the skin perfusion of the user and drug delivery is prevented. When a treatment is completed, the device 100 is deflated using the pump and a fluid outlet (or the fluid inlet (12) in reverse).

In one aspect, one or more perfusion sensors are placed on the surface of the plurality of sockets 108 forming the at least one primary bladder 5, which are in contact with the skin of the wearer. The inputs from the perfusion sensors can be used to modulate the level of compressive pressure applied by the at least one bladder 5 to the wearer.

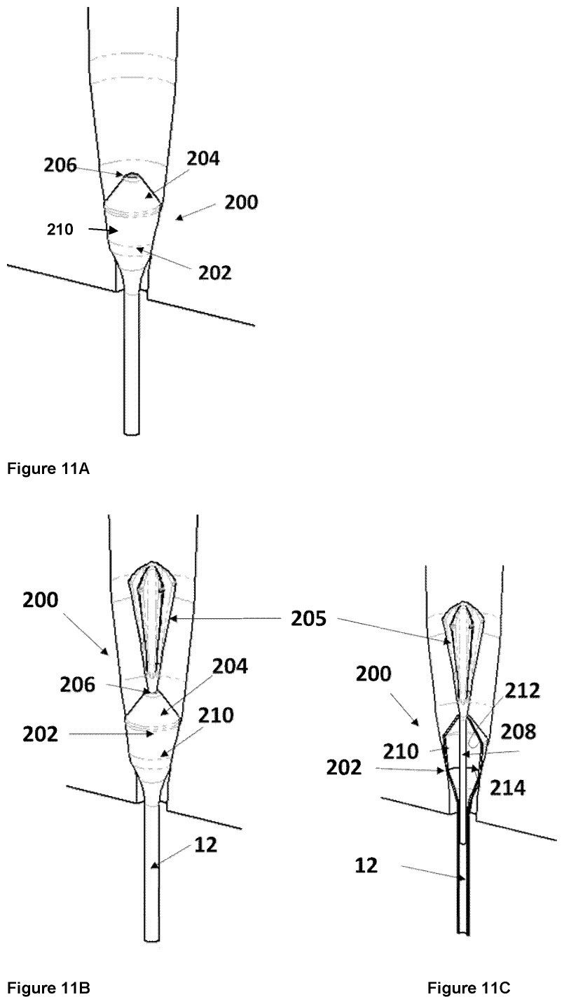

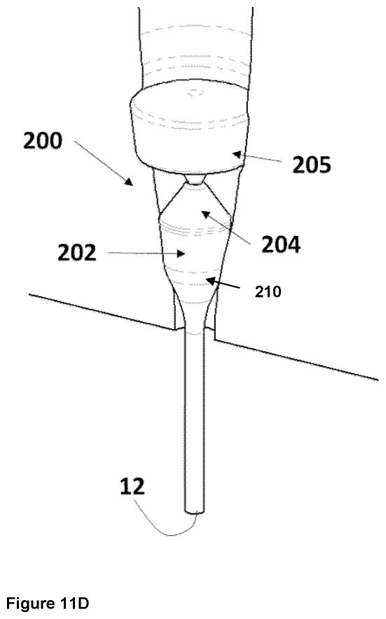

Referring now to FIG. 10 and FIG. 11, there is illustrated a general embodiment of a pneumatic compression device of the present invention for use within a cavity of a subject. Where elements of each embodiment are shared, the same reference numerals are used. Specifically, FIG. 10A illustrates a perspective view of the pneumatic compression device of the present invention in situ within the vaginal cavity of a subject and is generally referred to by reference numeral 200. The device 200 comprises at least one primary bladder 205 and an applicator 202 comprising an outer shell 210 and an inner shell 212 which forms an enclosure 214 that houses the at least one primary bladder 205, and a cap 204 that sits atop the applicator 202. The applicator 202 delivers the at least one primary bladder 205 to the tissue target when in an internal cavity, such as in the vaginal cavity as per FIG. 10 and FIG. 11. When placed correctly in the cavity by the user, the at least one primary bladder 205 is pushed from the enclosure 214 by a support axis 208 (see FIG. 10D) to within the confines of the cavity. The cap 204 is in communication with the support axis 208 and disengages from the end of the applicator 202 when activated (see FIG. 10B and FIG. 10D). When the at least one primary bladder 205 is outside of the enclosure 214 of the applicator 202, the at least one primary bladder 205, or a plurality of primary bladders 205, is inflated using a pump through the inlet 12, via a control system, as described above for devices 1 and 100, such that the at least one primary bladder 205, or a plurality of primary bladders 205, expand to apply compression against the walls of the cavity (see FIG. 100 and FIG. 10D).

The at least one primary bladder 205 inflates and incrementally applies compressive pressure against the skin of the wearer. The device 200 is programmable to remain inflated until the pressure reduces the tissue perfusion of the user and drug delivery is prevented. When a treatment is completed, the device 200 is deflated using the pump and a fluid outlet (or the fluid inlet (12) in reverse).

In one aspect, one or more perfusion sensors are placed on the surface of the at least one primary bladder 205, or the plurality of primary bladders 205, which are in contact with the tissue of the cavity. The inputs from the perfusion sensors can be used to modulate the level of compressive pressure applied by the at least one primary bladder 205, or a plurality of primary bladders 205, to the user.

Referring now in more detail to FIG. 11, the cap 204 that sits atop the applicator 202 further comprises an aperture 206. The aperture 206 is configured to guide the at least one primary bladder 205 from the enclosure 214 to the cavity. When a user acts on the support axis 208, the at least one primary bladder 205 exits the aperture 206 and into the confines of the cavity (see FIG. 11B). The cap 204 is disengaged from the support axis 208 and remains in communication with the top of the applicator 202. The support axis 208 remains engaged with the at least one primary bladder 205 (see FIG. 11C). When the at least one primary bladder 205 is outside of the enclosure 214 of the applicator 202, the at least one primary bladder 205, or a plurality of primary bladders 205, is inflated using a pump through the inlet 12, via the control system, as described above for devices 1 and 100, such that the at least one primary bladder 205, or a plurality of primary bladders 205, expand to apply compression against the walls of the cavity (see FIG. 11D).

The at least one primary bladder 205 inflates and incrementally applies compressive pressure against the skin of the wearer. The device 200 is programmable to remain inflated until the pressure reduces the tissue perfusion of the user and drug delivery is prevented. When a treatment is completed, the device 200 is deflated using the pump and a fluid outlet (or the fluid inlet (12) in reverse).

In one aspect, one or more perfusion sensors are placed on the surface of the at least one primary bladder 205, or the plurality of primary bladders 205, which are in contact with the tissue of the cavity. The inputs from the perfusion sensors can be used to modulate the level of compressive pressure applied by the at least one primary bladder 205, or a plurality of primary bladders 205, to the user.

Materials and Methods

The device 1,100,200 is operated by the wearer in the clinical setting as set out below.
1. The device 1,100,200 is provided in a loose, unfastened fashion.
2. For the scalp, the clinician or user places the device 1 loosely over the head of the subject and straps the device 1 in place using the securing mechanism 10 and the secondary bladder 11, the first fastening system 16 or the second fastening system 17, or a combination thereof.
3. The device 1 is switched on by pressing the on button 14 at the rear of the device 1. The device 1 then begins to inflate, and the status light 13 indicates that the device 1 is switched on and the at least one primary bladder 5 is beginning to inflate.
4. When the device 1 reaches the target pressure application, the status light 13 indicates that the device 1 is now in “active” mode. The device 1 will stay active for a pre-programmed period of time.
5. For the hand or foot, the clinician or user places the device 100 loosely over the hand or foot of the subject and straps the device 100 in place using the first securing member 2, the second securing member 102 or the third fastening member 103, or a combination thereof.
6. The device 100 is switched on by pressing a button on a control system that incorporates a pump electronically coupled with a pressure regulating system/sensor. The pump then begins to inflate the at least one primary bladder and a status light on the control system indicates that the device 100 is switched on and the at least one primary bladder 5 is beginning to inflate.
7. When the device 100 reaches the target pressure application, the status light indicates that the device 100 is now in “active” mode. The device 100 will stay active for a pre-programmed period of time.
8. For the cavity, the clinician or user places the device 200 within the cavity.
9. The device 200 is switched on by pressing a button on a control system that incorporates a pump electronically coupled with a pressure regulating system/sensor. The pump then begins to inflate the at least one primary bladder 205, and a status light on the control system indicates that the device 200 is switched on and the at least one primary bladder 205 is beginning to inflate.
10. When the device 200 reaches the target pressure application, the status light indicates that the device 200 is now in “active” mode. The device 200 will stay active for a pre-programmed period of time.
11. The clinician can then begin to operate the chemotherapy infusion process.
12. Once the chemotherapy infusion is complete, the patient is free to leave the clinical setting while wearing the device 1,100,200.
13. Once the active chemotherapeutic period is over, the status indicator light will show that the treatment is complete. The control element 9 or control system further comprises a timing system that can be set to a particular amount of time that the device 1,100,200 is inflated to apply pressure to the scalp/hand/foot/cavity of the user. The timing system can be set to the amount of time the chemotherapeutic treatment is being administered, or the control element 9 or control system can connect to the chemotherapeutic treatment delivery device (either physically connected via a plug-in-and-play wiring set up or wirelessly) and mirror the time the treatment will take. When the time period is complete, user can either then press the off button (14) or the off button (14) will automatically be disengaged by the control element 9 or control system, which triggers the pump to draw the fluid out of at least one primary bladder (or from the plurality of bladder compartments 5,205) and expel the fluid through the vent to release the pressure slowly from the device 1,100,200.
14. Once the pressure is released, the device 1,100,200 completes a shutdown procedure, and the user is then free to unfasten or remove the device 1,100,200 and store it.
15. The user brings the device 1,100,200 to clinic at their next appointment.

### CONCLUSION

The device 1,100,200 can be worn before, during and after chemotherapy treatment for a defined period. The projected wearing time is up to about 30 minutes before chemotherapy infusion, constant wearing during chemotherapy infusion and for a further 60-120 minutes, preferably 90 minutes, after infusion is completed. The key benefit of this solution is that the subject can leave the infusion ward while wearing the device 1,100,200, instead of having to spend the additional time after infusion treatment has finished in the infusion centre wearing the device 1,100,200.

The device 1,100,200 of the claimed invention is an elegant, comfortable, and portable solution to preventing hair loss during chemotherapy. The device 1,100,200, which is designed to fit into the existing clinical workflow, avoids the capital cost, infection control and patient discomfort issues associated with existing products.

The use of pneumatic bladders in the device 1,100,200 inherently expand to fill the empty space around them. This is a highly advantageous property in the device 1,100,200 because it means given enough room to expand, the bladder system will expand to fit any shape of head, hand, foot, or cavity, and will apply the same pressure equally to every surface it contacts.

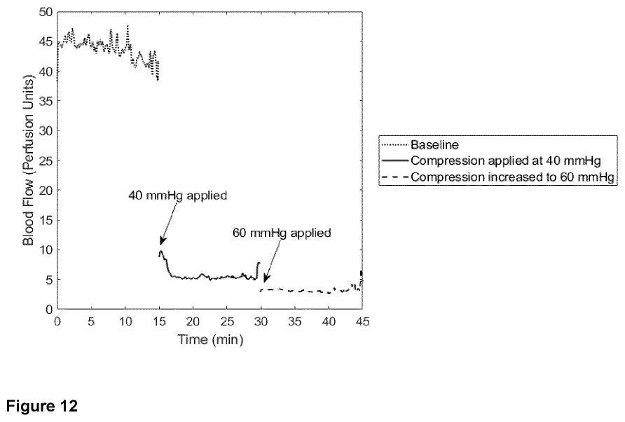

The device 1,100,200 modulate drug delivery at the microvascular level with low levels of pressure to avoid any issues with lack of blood flow to a large portion of the body. This is illustrated in FIG. 12, where the reduction in tissue perfusion at the target site from a “baseline” resting state to a significantly lower level is demonstrated when compression is applied using the device 1. This graph shows the average reduction in tissue perfusion at a depth of 1.5 mm (as measured using a Laser Doppler Flowmeter) at the target site while using the device 1 across eight healthy human volunteers. The existing devices of the prior art use vasoconstriction induced by cryogenics, and existing compression devices mainly focus on increasing blood flow.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

1. A pneumatic compression device (1,100) for use in preventing or treating chemotherapy-induced alopecia or chemotherapy-induced peripheral neuropathy (CIPN) in a subject receiving chemotherapy treatment, the device (1,100) comprising: an attachment member (2), a first layer (3) connected to the attachment member (2), at least one primary bladder (5); and a fluid inlet (12), wherein the at least one primary bladder (5) is configured to inflate with a fluid at ambient temperature, and which exerts a compressive pressure to the non-treated area of the subject when the bladder (5) is inflated minimising blood perfusion to prevent chemotherapy delivery to the non-treated area.

2. The pneumatic compression device (1) of claim 1, further comprising a second layer (4) and an outer mesh layer (6) surrounding the second layer (4).

3. The pneumatic compression device (1) of claim 1 or claim 2, further comprising an inner membrane (7), the inner membrane (7) forms an inner lining of the device (1), nestling between the second layer (4) and the tissue of the subject.

4. The pneumatic compression device of any one of the preceding claims, further comprising a secondary bladder (11), wherein the secondary bladder (11) is configured to attach to the attachment member (2) such that the secondary bladder (11) comes in contact with the tissue of the subject.

5. The pneumatic compression device (1) of any one of the preceding claims, further comprising a first fastening system (16) configured to prevent the outward movement of the outer mesh layer (6).

6. The pneumatic compression device (1) of claim 5, further comprising a second fastening system (17) adapted to tighten the attachment member (2) to the subject.

7. The pneumatic compression device (1) of any one of claims 2 to 6, wherein the at least one primary bladder (5) is attached along its inner edge (5a) to the second layer (4), and along its perimeter surface (5b) to the first layer (3) and along its lower edge surface (5c) to the attachment member (2).

8. The pneumatic compression device (1) of any one of the preceding claims, further comprising at least one adjustable strap (10) to secure the device (1) under, around or over the chin of the subject.

9. The pneumatic compression device (100) of claim 1, wherein the at least one primary bladder (5) is configured to have a plurality of sockets (108) to accommodate the fingers of the hand or the toes of the foot of the subject.

10. The pneumatic compression device (100) of claim 1 or claim 9, wherein the at least one primary bladder (5) is configured to be a single socket that accommodates the whole hand or the foot of the subject.

11. The pneumatic compression device (100) of any one of claim 1, 9 or 10, further comprising a first securing member (102) to secure the device (100) in place.

12. The pneumatic compression device (100) of any one of claim 1, 9, 10 or 11, further comprising a second securing member (103) to secure the device (100) in place.

13. The pneumatic compression device (1,100) of any one of the preceding claims, wherein the attachment member (2), the first fastening system (16), second fastening system (17), the first securing member (102), or the second securing member (103) can be used with greater force to tighten the device (1,100) against the surface of the scalp, foot, or hand through a drawstring mechanism.

14. A pneumatic compression device (200) for use in preventing or treating chemotherapy-induced infertility in a subject receiving chemotherapy treatment, the device (200) comprising: at least one primary bladder (205), an applicator (202) adapted to accommodate the at least one primary bladder (205), and a cap (204) configured to contain the at least one primary bladder (205) within the applicator (202) when deflated, wherein the at least one primary bladder (205) located within the applicator (202) is configured to inflate with air or a gas at ambient temperature, and which exerts a compressive pressure when the bladder (205) is inflated.

15. The pneumatic compression device (200) of claim 14, further comprising an outer shell (210) and an inner shell (212), which combine to form an enclosure (214) that accommodates the at least one primary bladder (205).

16. The pneumatic compression device (200) of claim 14 or claim 15, further comprising a support means (208) housed internally in the applicator (202) and in communication with the cap (204).

17. The pneumatic compression device (200) of claim 16, wherein the support means (208) is surrounded by the at least one primary bladder (205).

18. The pneumatic compression device (200) of any one of claims 14 to 17, wherein the cap (204) is reversibly connected to the applicator (202) and is released from the applicator (202) when the at least one primary bladder (205) is inflated.

19. The pneumatic compression device (200) of any one of claims 14 to 17, wherein the cap (204) is fixed to the applicator (202) and further comprises an aperture (206) through which the at least one primary bladder (205) is pushed through prior to inflation.

20. The pneumatic compression device (1,100,200) of the preceding claims, further comprising a control element (9).

21. The pneumatic compression device (1,100,200) of claim 20, wherein the control element (9) comprises a pump, a pressure sensor, and a tissue perfusion sensor.

22. The pneumatic compression device (1,100,200) of claim 21, wherein the pressure sensor is in communication with the at least one primary bladder (5,205).

23. The pneumatic compression (1,100,200) device of any one of the preceding claims, wherein the at least one primary bladder (5,205) further comprises a plurality of bladder compartments, each bladder compartment operating independently of each other.

24. The pneumatic compression device (1,100,200) of any one of claims 1 to 22, wherein the at least one primary bladder (5,205) further comprises a plurality of bladder compartments, each of the plurality of bladder compartments are in fluid communication with a bladder compartment juxtaposed it.

25. The pneumatic compression device (1,100,200) of claim 23 or claim 24, wherein the at least one primary bladder (5,205) or the plurality of bladder compartments are connected to the pump, the pump having a two-way valve system to control the inflow and outflow of the fluid.

26. The pneumatic compression device (1,100,200) of any one of the preceding claims, wherein when activated, the device (1,100,200) applies a compressive pressure of between about 20 mmHg to about 100 mmHg to the subject.

27. The pneumatic compression device (1,100,200) of claim 26, wherein the compressive pressure being applied is between about 25 mmHg and about 75 mmHg.

28. The pneumatic compression device (1,100,200) of claim 26 or claim 27, wherein the compressive pressure being applied is between about 40 mmHg and about 60 mmHg.

29. The pneumatic compression device (1,100,200) of any one of the preceding claims, wherein the compressive pressure is applied incrementally.

30. The pneumatic compression device (1,100,200) of claim 29, wherein the compressive pressure is applied uniformly and/or non-uniformly.

31. The pneumatic compression device (1,100,200) of any one of the preceding claims, further comprising a foam layer inserted within the at least one primary bladder (5,205) or one or more of the plurality of bladders; wherein the foam layer exerts a reaction force through the bladder onto the scalp, foot, hand, or within a cavity when air is withdrawn incrementally creating a vacuum inside the at least one primary bladder (5,205) or one or more of the plurality of bladders.

32. The pneumatic compression device (1,100) according to any one of the preceding claims, wherein a fluid or a solid which is viscous at ambient pressure and a solid form at pressures lower than atmospheric pressure, is inserted into the at least one primary bladder (5) or one or more of the plurality of bladders, wherein when pressure within the at least one primary bladder (5) decreases, the fluid or the sold hardens, creating a modifiable compressive force against the tissue.

33. The pneumatic compression device (1,100,200) of any one of the preceding claims, wherein the at least one primary bladder (5,205) or the plurality of bladders, are inflated are to a pre-determined pressure level below the target therapeutic pressure level prior to the user using the device (1,100,200).

34. The pneumatic compression device (1,100,200) of any one of the preceding claims, wherein the at least one primary bladder (5,205) or the plurality of bladders are provided at a pre-determined pressure level and are not further inflated.

35. The pneumatic compression device (1,100,200) of any one of the preceding claims, wherein the attachment member (2) further comprises at least one status indicator light (13).

36. A method of controlling drug delivery via the blood vessels to a target site on the body, the method comprising:
mechanically compressing the skin/surface tissue at a target tissue site at a predetermined compressive force to occlude the upstream blood vessels;
maintaining the compressive force for a predetermined period; and
releasing the compressive force applied to the tissue at the target site at a pre-determined rate and in a pre-determined geometric pattern.

37. The method of claim 36, wherein the compressive force is applied to the tissue site upstream of the intended effect site.

38. The method of claim 36 or claim 37, wherein the compressive force is applied to the skin/surface tissue at the target site at about 20 mmHg to about 100 mmHg.

39. The method of any one of claims 36 to 38, wherein the compressive force applied is configured to mechanically occlude the microvasculature close to the surface of the tissue and local arterial supply vessels.

40. The method of any one of claims 36 to 39, wherein the compressive pressure is applied uniformly across the tissue site to be treated.

41. The method of any one of claims 36 to 40, wherein the compressive force is maintained for a period of between 30 minutes and 7 hours.

42. The method of any one of claims 36 to 41, wherein the compressive force is applied at different areas of the tissue.

43. The method of any one of claims 36 to 42, wherein the compressive force is applied in a pulsatile fashion.

44. A method of controlling drug delivery to a target site on the body, followed by a period of reactivating blood flow to ensure long-term tissue viability, the method, comprising:
mechanically compressing the skin/surface tissue at the target site with a predetermined compressive force to cause occlusion of the local microvasculature;
maintaining microvasculature occlusion for a predetermined period of time such that drugs circulating through the vascular system do not reach the target site or site downstream of the target site;
releasing the occlusion in a controlled manner at a pre-determined rate and in a pre-determined geometric pattern;
sensing a physiological parameter;
adjusting the mechanical compression applied to the tissue site based on the sensed physiological parameter; and
applying compressive forces in a pulsatile fashion against the skin tissue to promote blood flow allowing waste agents to be removed from the local vasculature.

45. The method of claim 44, wherein the sensed physiological parameter is the blood perfusion in the tissue site being treated.
